(19) United States
(12) Patent Application Publication
(10) Pub. No.: US 2003/0097177 A1
(43) Pub. Date: May 22, 2003

(54) Posterior chamber phakic lens

(76) Inventor: Son Trung Tran, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 09/990,710

(22) Filed: Nov. 21, 2001

(51) Int. Cl. A61F 2/16

(52) U.S. Cl. 623/6.47; 623/6.44

(57) ABSTRACT

A posterior chamber phakic lens made from an elastomeric, foldable, highly biocompatible material. The lens has a generally circular optic and a pair of integrally formed, plate-style haptics. The haptics project posteriorly from the optic. A plurality of openings are formed at the intersection of the optic and the haptics, the holes extending all the way through the lens. The distal tips of the haptics are split or forked so as to project anteriorly and posteriorly. The anterior arm is designed to fit against the posterior iris, and the posterior arm is design to be supported in the anterior ciliary sulcus. Such a construction provides for a stable lens once implanted in the eye, helps to avoid pupillary blockage and allows for improved aqueous flow around the natural lens.

### BACKGROUND OF THE INVENTION

This invention relates generally to the field of intraocular lenses (IOL) and, more particularly, to posterior chamber phakic IOLs.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

The optical power of the eye is determined by the optical power of the cornea and the crystalline lens. In the normal, healthy eye, sharp images are formed on the retina (emmetropia). In many eyes, images are either formed in front of the retina because the eye is abnormally long (axial myopia), or formed in back of the retina because the eye is abnormally short (axial hyperopia). The cornea also may be asymmetric or toric, resulting in an uncompensated cylindrical refractive error referred to as corneal astigmatism. In addition, due to age-related reduction in lens accommodation, the eye may become presbyopic resulting in the need for a bifocal or multifocal correction device.

In the past, axial myopia, axial hyperopia and corneal astigmatism generally have been corrected by spectacles or contact lenses, but there are several refractive surgical procedures that have been investigated and used since 1949. Barraquer investigated a procedure called keratomileusis that reshaped the cornea using a microkeratome and a cryolathe. This procedure was never widely accepted by surgeons. Another procedure that has gained widespread acceptance is radial and/or transverse incisional keratotomy (RK or AK, respectively). Recently, the use of photablative lasers to reshape the surface of the cornea (photorefractive keratectomy or PRK) or for mid-stromal photoablation (Laser-Assisted In Situ Keratomileusis or LASIK) have been approved by regulatory authorities in the U.S. and other countries. All of these refractive surgical procedures cause an irreversible modification to the shape of the cornea in order to effect refractive changes, and if the correct refraction is not achieved by the first procedure, a second procedure or enhancement must be performed. Additionally, the long-term stability of the correction is somewhat variable because of the variability of the biological wound healing response between patients.

Several companies are investigating implantable posterior chamber phakic IOLs, including the Staar ICL lens and the Medennium PRL lens. These and other posterior chamber phakic lenses are described in U.S. Pat. No. 4,769,035 (Kelman), U.S. Pat. No. 6,015,435 (Valunin, et al.) and U.S. Pat. No. 6,106,553 (Feingold), the entire contents of which being incorporated herein by reference. The clinic experience with commercially available posterior chamber phakic lenses has not been entirely satisfactory due to pupillary block, the need to accurately size the lens, unwanted rotation of the lens and the development of traumatic cataract.

Therefore, a need continues to exist for a safe, stable and biocompatible posterior chamber phakic intraocular lens.

### BRIEF SUMMARY OF THE INVENTION

The present invention improves upon the prior art by providing a posterior chamber phakic lens made from an elastomeric, foldable, highly biocompatible material. The lens has a generally circular optic and a pair of integrally formed, plate-style haptics. The haptics project posteriorly from the optic. A plurality of openings are formed at the intersection of the optic and the haptics, the holes extending all the way through the lens. The distal tips of the haptics are split or forked so as to project anteriorly and posteriorly. The anterior arm is designed to fit against the posterior iris, and the posterior arm is design to be supported in the anterior ciliary sulcus. Compressive forces exerted by the ciliary sulcus forces apart the anterior arm and the posterior arm. Such a construction provides for a stable lens once implanted in the eye, helps to avoid pupillary blockage and allows for improved aqueous flow around the natural lens.

Accordingly, one objective of the present invention is to provide a safe and biocompatible intraocular lens.

Another objective of the present invention is to provide a safe and biocompatible intraocular lens that is easily implanted in the posterior chamber.

Still another objective of the present invention is to provide a safe and biocompatible intraocular lens that is stable in the posterior chamber.

Still another objective of the present invention is to provide a safe and biocompatible intraocular lens that does not need highly accurate sizing.

These and other advantages and objectives of the present invention will become apparent from the detailed description and claims that follow.

### BRIEF DESCRIPTION OF THE DRAWING

FIG. 1 is an enlarged top plan view of the lens of the present invention.

FIG. 2 is an enlarged cross-sectional view of the lens of the present invention taken at line 2-2 in FIG. 1.

FIG. 3 is an enlarged side view of the lens of the present invention.

### DETAILED DESCRIPTION OF THE INVENTION

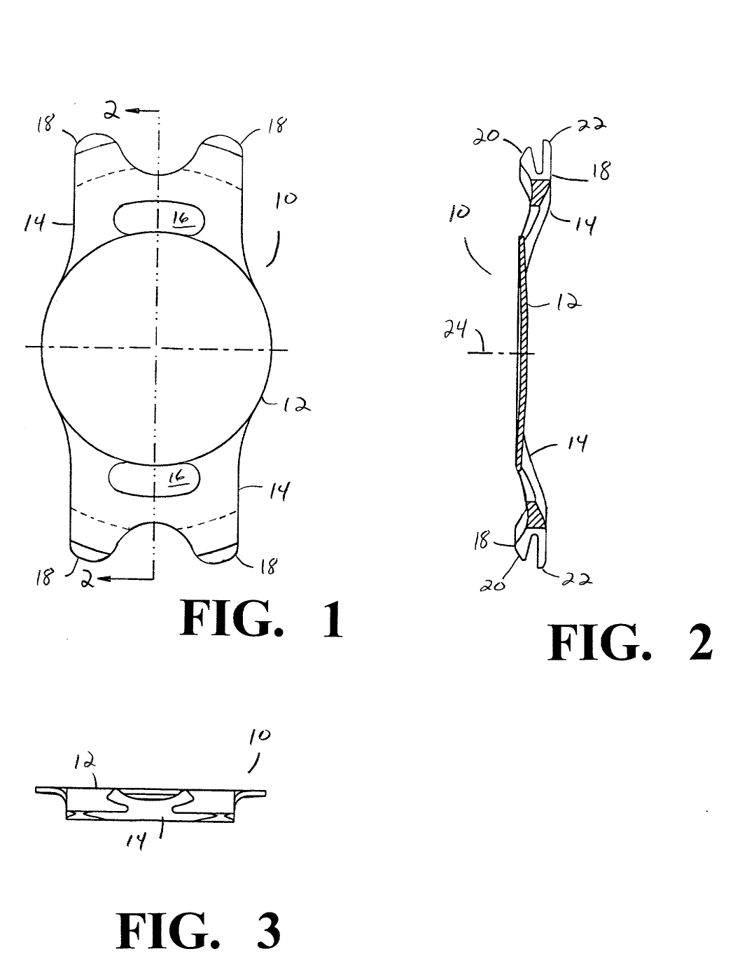

As best seen in FIGS. 1, 2 and 3, lens 10 of the present invention generally includes optic 12 and at least two plate-style haptics 14 integrally formed with optic 12. Optic 12 may be of any suitable size, such as between 4.5 mm and 6.5 mm in diameter, and may be biconcave, biconvex, concave/convex or any other suitable geometry. Optic 12 may also contain refractive or diffractive features, such features being well-known in the art. Lens 10 is preferably formed in any suitable overall length, for example, around 10.5 millimeters, for implantation in the posterior chamber in front of the natural lens, from a soft, foldable material such as a hydrogel, silicone or soft acrylic, such diameters and materials being well-known in the art. As best seen in FIGS. 2 and 3, haptics 14 project or vault posteriorly from optic 12, so as to locate optic 12 anteriorly of haptics 14 once implanted in an eye. At the intersection of haptics 14 and optic 12, haptics 14 contain openings 16 that extend all the way through lens 10. Although FIG. 1 illustrates openings 16 as being oval, one skilled in the art will recognize that openings 16 may be round, oval or any other suitable shape and of any suitable number. The vaulting of optic 12 anteriorly, along with openings 16, allow for increased aqueous flow around the natural lens and reducing pupillary blockage.

As best seen in FIG. 2, distal tips 18 of haptics 14 are split along a plane normal or perpendicular to optical axis 24 of optic 12 to form anterior arm 20 and posterior arm 22. Anterior arm 20 is sized and shaped so as to lodge against the posterior side of the iris once implanted in an eye. Posterior arm 22 is sized and shaped to lodge in the ciliary sulcus once implanted in an eye. Once implanted, compression of haptics 14 by the sulcus forces arm 20 anteriorly and forces arm 22 posteriorly, thereby helping to lock in or stabilize lens 10 and desensitizing the need to size lens 10 accurately. Although FIG. 1 shows haptics 14 having a pair of distal tips 18, one skilled in the art will recognize that other configurations of distal tips 18 may also be used.

This description is given for purposes of illustration and explanation. It will be apparent to those skilled in the relevant art that changes and modifications may be made to the invention described above without departing from its scope or spirit.

We claim:

1. An intraocular lens, comprising:
a) an optic;
b) at least two plate-style haptics connected to and vaulting posteriorly from the optic; and
c) at least one distal tip on the haptics, the distal tips being split into an anterior arm and a posterior arm.

2. The lens of claim 1 wherein the haptics are integrally formed with the optic.

3. The lens of claim 1 wherein the optic comprises a soft acrylic.

4. The lens of claim 1 wherein the optic comprises a hydrogel.

5. The lens of claim 2 wherein the lens comprises a soft acrylic.

6. The lens of claim 2 wherein the lens comprises a hydrogel.

7. An intraocular lens, comprising:
a) an optic;
b) a pair of plate-style haptics connected to and vaulting posteriorly from the optic; and
c) a pair of distal tips on the haptics, the distal tips being split into an anterior arm and a posterior arm.

8. The lens of claim 7 wherein the haptics are integrally formed with the optic.

9. The lens of claim 7 wherein the optic comprises a soft acrylic.

10. The lens of claim 7 wherein the optic comprises a hydrogel.

11. The lens of claim 8 wherein the lens comprises a soft acrylic.

12. The lens of claim 8 wherein the lens comprises a hydrogel.
